(19) United States
(12) Patent Application Publication
(10) Pub. No.: US 2024/0309066 A1
(43) Pub. Date: Sep. 19, 2024

(54) MEANS AND METHODS FOR THE TREATMENT OF CALCIUM CRYSTAL DEPOSITION DISEASES

(71) Applicants: UNIVERSITEIT MAASTRICHT, MAASTRICHT (NL); ACADEMISCH ZIEKENHUIS MAASTRICHT, MAASTRICHT (NL)

(72) Inventors: Tim Johannes Maria WELTING, MAASTRICHT (NL); Guus Gijsbertus Hubert VAN DEN AKKER, MAASTRICHT (NL); Leon Johannes SCHURGERS, MAASTRICHT (NL); Lodewijk Willem VAN RHIJN, MAASTRICHT (NL)

(21) Appl. No.: 18/576,049

(22) PCT Filed: Jun. 27, 2022
(86) PCT No.: PCT/EP2022/067584

§ 371 Date: Jan. 2, 2024

(30) Foreign Application Priority Data
Jul. 5, 2021 (EP) 21183609.3

(51) Int. Cl. C07K 14/78; A61K 38/00; A61P 19/02

(52) U.S. Cl. CPC C07K 14/78; A61P 19/02; A61K 38/00

(57) ABSTRACT

The invention is in the field of medical treatments, in particular the treatment of humans, more in particular humans with calcium deposition diseases. In one aspect, the invention provides polypeptides that prevent or decrease the precipitation of calcium crystals in the human body, also known as ectopic calcification. More in particular, the invention relates to a circular polypeptide consisting of an amino acid sequence according to SEQ ID NO: 1, wherein the amino acids are D-amino acids, pharmaceutical compositions comprising a polypeptide as described herein and their use in the treatment of a disease selected from the group consisting of osteoarthritis, frozen shoulder syndrome, heterotopic ossification, vascular calcification, kidney stones and calcinosis.

### FIELD OF THE INVENTION

The invention is in the field of medical treatments, in particular the treatment of humans, more in particular humans with calcium crystal deposition diseases. In one aspect, the invention provides polypeptides that prevent or decrease the precipitation of calcium-containing crystals in the human body, also known as ectopic calcification.

### BACKGROUND OF THE INVENTION

Abnormal calcium crystal deposition, also known as calcification of soft tissues is a major issue in a multitude of systemic diseases or chronic diseases such as atherosclerosis (1), kidney failure (2) and osteoarthritis (3). In the case of atherosclerosis and osteoarthritis, the importance of these calcium depositions was initially not recognized (4, 5), while this has been well recognized in kidney disorders (6). It has become increasingly clear that calcium-containing crystals, oftentimes difficult to detect in the form of micro particles, can indeed cause disease or at least exacerbate disease (7, 8). Pathological calcium depositions consist mostly of Basic Calcium Phosphate (BCP) and Calcium Pyrophosphate dihydrate (CPP).

Control over calcium crystal deposition is of vital importance in multicellular organisms, where high concentrations of metal ions and anions, such as inorganic phosphate (pi), can be found in the intra- and extracellular environment. Extracellular matrix components, such as collagens, can facilitate calcification by acting as a nucleation center (9). On the other hand, mineralization is crucial for bone development and homeostasis (10). Therefore, calcium ion levels are maintained within stringent boundaries (between 1.15 and 1.33 mmol/L) in the blood stream by the hormones PTH and Calcitonin, which is crucial for muscle contraction and nerve impulse generation (11). Total calcium levels range between 2.2 and 2.7 mM, wherein about 45% is ionic, around 45% is protein bound and 10% is complexed (12). Phosphate levels are known to fluctuate due to dietary intake (between 0.8 mM and 1.5 mM) and only a small portion (about 10 to 15%) is protein bound (13). Endogenous inhibitors of calcification include inorganic pyrophosphate (ppi), which is stimulated by Ectonucleotide Pyrophosphatase/Phosphodiesterase 1 (Enpp1; (14)), Ankyrin 1 (Ank1; (15)) and ATP Binding Cassette subfamily C member 6 (Abcc6; (16)), as evidenced by murine knock-out animal studies. Other important inhibitors of calcification include Matrix Gla protein (MGP), Gla-rich protein (GRP), Klotho (KL) and Alpha-2-HS-Glycoprotein (AHSG, also known as Fetuin A).

Despite the availability of several endogenous inhibitors of calcification, no effective composition exists for curing or preventing calcium crystal deposition diseases.

### LEGEND TO THE FIGURES

FIG. 1: Inhibitory effect of peptide according to SEQ ID NO: 1 and bFetuin on calcium precipitation over a broad concentration range in vitro. Dilution series in steps of 10 (total 10¹⁵) of the peptide in a calcium precipitation assay (2 hours). Hundred percent precipitation is obtained without any addition of bFetuin or peptide 1 (dashed line).

FIG. 2: Electron microscopy images obtained from precipitates formed after two hours in the absence (left panel) or presence (right panel) of the peptide (20 μM). Precipitates formed in the presence of bFetuin is shown in the middle panel. Precipitation inhibition of ˜50% was confirmed by absorption measurements prior to sample processing (dialysis). The magnification of the scanning electron microscope was 35.000×. Bar represents 5 micrometer. Middle panel: 0.5 mg/ml bFetuin, right panel: 20 μM peptide 1 added.

FIG. 3: Inhibition of cellular calcification of human articular chondrocytes, vascular smooth muscle cells and bone marrow stromal cells by the peptide according to SEQ ID NO: 1. Representative phase-contrast microscopy images of in vivo cell assays are shown.
A) Human articular chondrocytes (HACs) were stimulated for 7 days with 1 mM ATP and with or without 20 μM of the peptide. Results were obtained from three chondrocyte donors with three biological replicates each.
B) Vascular smooth muscle cells (VSMC) were stimulated for 7 days with 4.5 mM ionic calcium in the presence or absence of 20 μM of peptide. Results were obtained from three VSMC donors with three biological replicates each.
C) BMSCs were differentiated for 21 days in osteogenic differentiation medium containing beta-glycerophosphate (BGP) in the pre- or absence of 2.1 μM of peptide. BMSC; one donor with four biological replicates was used.
Left panels: phase-contrast images were obtained at the final day of each experiment. Calcifications can be seen as black dots. One representative image is shown per condition. Scale bars indicate 200 μm.
Right panels: quantification of total calcium precipitation per well measured with the Randox total calcium assay and normalized to total protein. For each donor the positive control was normalized to 100%. Statistical comparisons were made using a student t-test. *=p. value<0.05.

FIG. 4: Intra-articular injection of the peptide ameliorates cartilage histopathology and improves animal mobility in a rat osteoarthritis model. Osteoarthritis was induced by introducing a surgical tear of the medial meniscus and transection of the collateral ligament (N=40). Bi-weekly intra-articular injections of 50 μl peptide with a concentration of 20 μM or 50 μl vehicle (0.9% NaCl) were performed (N=20 per group). Necropsy was performed at day 28, followed by histological analyses according to the OARSI scoring system.

A) Representative toluidine blue stained tissue sections from the best, average and worst histological appearance. Cartilage tissue is stained in dark blue.
B) The OARSI score was obtained by multiplying grade and stage of cartilage damage per animal by two blinded observers. Statistical comparison was done with a two-tailed t-test. *=p. value<0.05.
C) Gait analyses score at day 20 of the experiment. Four measurements per animal were performed and each dot represents the average of one animal in the graph. Statistical significance between groups was determined with a Mann-Whitney U test. *=p. value<0.05.

### SUMMARY OF THE INVENTION

The invention relates to a circular polypeptide consisting of an amino acid sequence according to SEQ ID NO: 1, wherein the amino acids are D-amino acids. The invention also relates to a pharmaceutical composition comprising such a polypeptide and a pharmaceutically acceptable carrier as well as its use in the treatment of a disease. More in particular, the invention relates to a polypeptide as described herein for use in the treatment of a disease selected from the group consisting of osteoarthritis, frozen shoulder syndrome, heterotopic ossification, vascular calcification, kidney stones and calcinosis.

### DETAILED DESCRIPTION OF THE INVENTION

We chemically synthesized a circular polypeptide consisting of 30 D-amino acids with the sequence: LIYRQPNCDDPETEEAALVAIDYIAPHGPG (hereinafter Peptide No: 1, PEPTIDE, peptide, or Peptide according to SEQ ID NO: 1) wherein the amino acid D-Leucine (L) at position 1 is chemically linked to the amino acid D-Glycine (G) at position 30, thereby forming a circular polypeptide. The terms “polypeptide” and “peptide” are used interchangeably herein.

To evaluate the inhibitory capacity of the peptide according to SEQ ID NO: 1, we utilized a well-established in vitro calcium phosphate precipitation assay (17-19) as described in example 2. As a positive control, we used bovine Fetuin (bFetuin) in a concentration of 1 mg/ml (20.6 μM), which was found to completely inhibit precipitation.

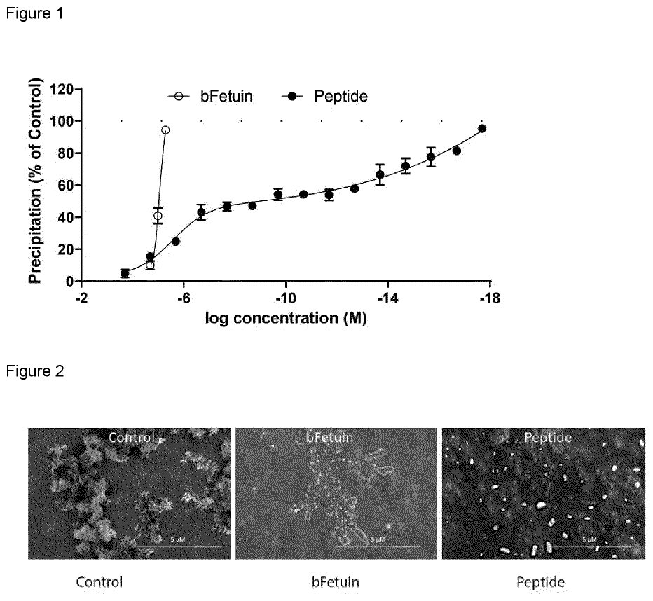

We set out to dilute the peptide until it was no longer effective. The peptide according to SEQ ID NO: 1 showed a positive dose-response effect upon increasing concentrations of peptide. Bovine fetuin (bFetuin) lost its inhibitory effect between 1 and 10 μg/ml (20.6-200.6 nM), whereas the peptide according to SEQ ID NO: 1 remained active until the attomolar concentration range (FIG. 1).

We found that even 20.6 aM, but not 2.6 aM was able to statistically significantly inhibit precipitation compared to control (FIG. 1). This 20.6 aM concentration was estimated to correspond with ˜1.250 peptide molecules in a 100 μl reaction volume.

In summary, the peptide according to SEQ ID NO: 1 inhibits in vitro precipitation of calcium phosphate more efficiently than bFetuin. It is concluded that the peptide according to SEQ ID NO: 1 is more effective than bFetuin in preventing calcium crystal deposition in an accepted in vitro model of calcium crystal deposition diseases.

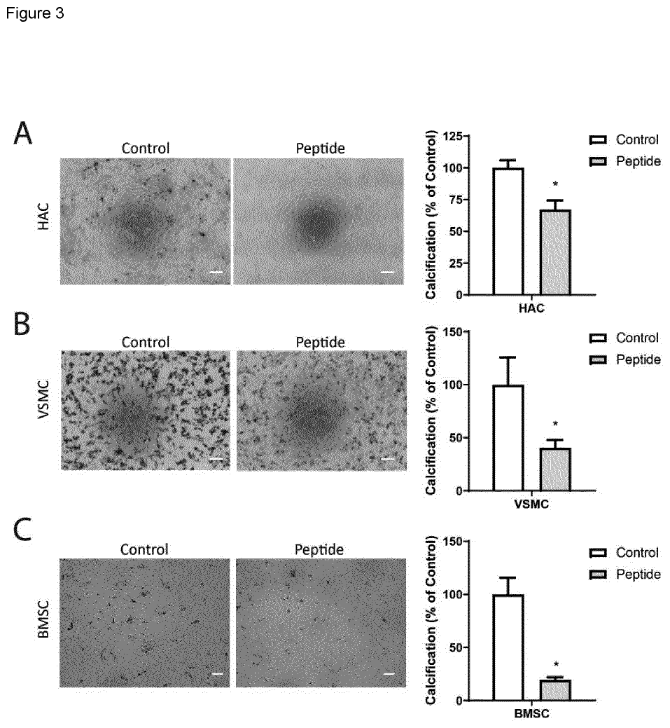

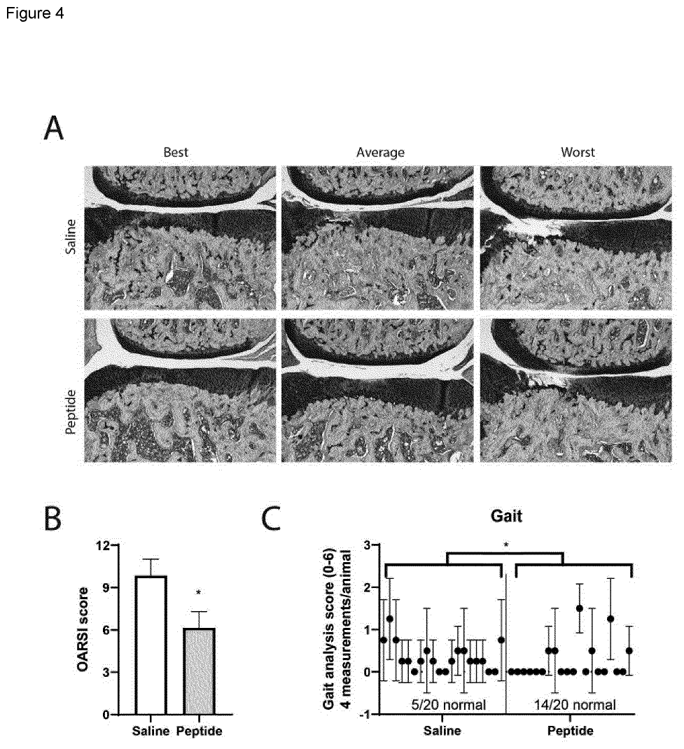

Next, we analyzed the precipitates that were formed after two hours in the in vitro assay in the presence or absence of bFetuin or the peptide according to SEQ ID NO: 1 by scanning electron microscopy (Example 3). In the control we found relatively large (˜1 μm) and small (50-200 nm) particles (FIG. 2; left panel). The larger particles had distinct crystalized edges, which are typical for BCP crystal formation (20). The particles obtained after contact with bFetuin had an entirely different morphology that lacked these sharp edges (FIG. 2; middle panel). Particles also appeared to be somewhat smaller (in the range of about 0.5 μm). The particles obtained with the peptide according to SEQ ID NO: 1 appeared even smaller and more compact. They had triangular, squared or in rare cases penta- or hexagonal morphologies (FIG. 2; right panel). The smallest discernable particles from the peptide treated sample had a size of about 67 nm±7 (Mean±SD, n=4), which resemble primary CPP (<100 nm). The particles obtained using the peptide were susceptible to dissolution by the electron beam at high magnification (>35.000×), which was not the case for particles from the control sample. It is known that bFetuin prevents CPP growth by covering the outer surface. Without wanting to be bound by theory, we therefore hypothesized that the peptide also interacts with the calcium phosphate particles.

To test this, particles were allowed to form for 2 or 24 hours in the presence or absence (control) of bFetuin or the peptide according to SEQ ID NO: 1. and subsequently harvested by centrifugation, washed and dissolved in acid, followed by total calcium, phosphate and protein measurements in the pellet and the supernatant.

The amount of calcium in the control pellets remained similar between two and twenty-four hour reaction times.

In pellets obtained with bFetuin and the peptide, the amount of precipitated calcium increased as a function of time from <1% at 2 hours incubation to more than 2% at 24 hours of incubation. This was still significantly lower than in the control without peptide or bFetuin (26% and 28% at 2 and 24 hours respectively).

Phosphate in the pellet increased from 2 to 24 hours from 2% to 14% in control pellets. Addition of bFetuine or peptide significantly reduced phosphate content at 24 hours to 9%. Finally, total protein content determination established that both bFetuin and the peptide were incorporated in the pellet. Up to 4% of bFetuin and 22% of the peptide was incorporated in the pellet. Taken together, these analyses demonstrate that both bFetuin and the peptide according to SEQ ID NO: 1 are incorporated in the calcium phosphate particles and change their morphology and composition.

To test if the peptide also showed in vivo anti-calcification properties, we tested its effect in three different well-established cellular calcification models: an ATP-induced human articular chondrocyte calcification model (HAC), a calcium-induced vascular smooth muscle cell (VSMC) calcification model and a beta-glycerophosphate-induced bone-marrow-derived mesenchymal stem cell (BMSC) osteogenic differentiation model (Example 6, (21-23)).

After 7 days of ATP stimulation of chondrocytes, we found 22-33% inhibition of calcification with the peptide when compared to the control calcification which is defined here as the maximum precipitation obtainable in the presence of ATP. (FIG. 3A).

In the 7 days VSMC calcification model, we found 58-61% inhibition of calcification with the peptides (FIG. 3B).

Finally, in the three weeks BMSC differentiation model, the peptide showed strong inhibition (81%) of calcium precipitation (FIG. 3C). The peptide treatment effect was clearly visible in phase-contrast images (FIG. 3C).

In conclusion, the peptide inhibits calcification in vivo in three different cell models.

We also evaluated the effect of bi-weekly intra-articular injection of the peptide in a rat osteoarthritis model (FIG. 4). Osteoarthritis was induced with a surgical tear of the medial meniscus and transection of the collateral ligament (N=40). Bi-weekly intra-articular injections of 50 μl peptide with a concentration of 20 μM or 50 μl vehicle (0.9% NaCl) were performed (N=20 per group). Necropsy was performed at day 28, followed by histological analyses according to the OARSI scoring system (24).

At 28 days post-surgery, we found that the peptide significantly reduced the histopathological OA score by 38% in the rat knee joints treated with the peptide (FIG. 4B) compared to treatment with vehicle. Most remarkably, there was a large and significant improvement in gait score after peptide treatment with 16/20 animals having a normal gait, whereas only 5/20 animals had a normal gait in the vehicle control treatment group (FIG. 4C).

In summary, we found that intra-articular injection of the peptide ameliorates the extent of osteoarthritis pathology and improves animal mobility in a rat osteoarthritis model.

### EXAMPLES

### Example 1: Peptide Synthesis

Lyophilized peptide was synthesized by PepScan (NL). Batches were generated with >90% purity. Purification was performed with pHPLC, whereas mass and UV profile were evaluated by MS-UPLC analysis (C18 RP-HPLC column).

### Example 2: In Vitro Calcium Phosphate Precipitation Assay

Calcium precipitation assays were performed essentially as described before with minor adaptations (19). 2.4 mM ionic calcium (0.1M stock) was added to a 50 mM Tris/HCL buffer (pH 7.4) in 1.5 ml Eppendorf tubes. Peptide or bFetuin were added and incubated for 15 minutes at room temperature. Subsequently, 1.6 mM phosphate buffer (0.1M stock) was added and the mixture was incubated for 120 minutes at 37° C. A positive control without peptide or bFetuin was taken along twice, once at the start and once at the end of a series, to verify that timing did not affect the result.

Data were normalized to the first positive control (normalized to 100%) in each experiment. As an internal reference for precipitation inhibition, we used 1 mg/ml, 0.5 mg/ml and 0.25 mg/ml bovine Fetuin (bFetuin, Sigma-Aldrich, #F2379) in each experiment. After 2 hours, samples were transferred to cuvettes (1.0 ml reactions) and absorbance was measured at A620 in a plate reader with a plate reader (Biorad). Each condition consisted of three biological replicates.

### Example 3: Scanning Electron Microscopy

After two hours of in vitro calcium phosphate precipitation, the reactions were stopped by transferring samples to dialysis membranes. After overnight dialysis, samples were freeze-dried, mounted on stubs and gold coated to enhance contrast prior to SEM investigation (25). Images were obtained at 10.0 kV and 0.40 nA.

### Example 4: Calcium Assay

Quantification of deposited calcium was carried out using a calcium determination kit (Randox, London, United Kingdom) according to the manufacturer's instruction, after hydrolysis of mineral deposits in 0.1M HCl. Calcium measurements were normalized to protein content using micro DC Protein Assay (Termo Scientifc, Bleiswijk, the Netherlands). To be able to measure protein content, an equal amount of 0.1M NaOH was used to neutralize the acid and 0.1% SDS (final concentration) was added to lyse cells in the cellular calcification assays.

### Example 5: Phosphate Assay

Quantification of deposited phosphate was carried out using a phosphate colorimetric assay (Sigma, MK030) according to manufacturer's instruction after solubilisation of precipitates in 0.1M HCL.

### Example 6: Cellular Calcification Models

Human primary smooth muscle cells (VSMCs) were derived from tissue explants from patients undergoing surgery. Tissue was dissected into ±5 mm² pieces and isolated as described before (26). In brief; cells were cultured in M199 with 20% FCS, 1% Pen/Strep (Gibco). Success of the isolation was determined by immunofluorescence staining for positive expression of alpha-smooth muscle actin (αSMA), smooth muscle protein 22-alpha (SM22α), phosphorylated myosin light chain 2 (pMLC) and absence of S100 C Calcium Binding Protein 4 (S100A4). Cells at passages between 5-8 were used for experiments and 10.000 cells per cm² were seeded for experiments. After 24 hours, medium was changed to calcification medium (growth medium with 5.4 mM Calcium or 5.4 mM calcium with peptide). Media were refreshed every second or third day until visual confirmation of calcification at day 7.

Human primary chondrocytes (HACs) were isolated from articular cartilage from total knee replacement surgery in end-stage osteoarthritis patients as left-over material after written informed consent (METC 2017-0183). Cells were cultured in DMEM/F12 with 10% FCS, 1% Pen/Strep (Gibco) and 1% non-essential amino acids (Gibco). Cells at passages 3-5 were used for experiments and 30.000 cells per cm2 were seeded for experiments. After 24 hours, medium was changed to calcification medium (growth medium with 1 mM ATP or 1 mM ATP with peptides at indicated concentrations). Media were refreshed every second or third day until visual confirmation of calcification at day 7.

Bone marrow derived stromal cells (BMSCs) were isolated from human bone marrow aspirates from the iliac crest of young individuals (METC 08-4-056). Cells were cultured in DMEM high glucose (Thermo Fisher) with 10% FCS and 1% Pen/strep (Gibco). Osteogenic differentiation was achieved with culture medium, supplemented with ascorbic acid 50 μg/ml, 100 nM dexamethasone and 10 mM beta-glycerophosphate. Media were refreshed every second or third day until visual confirmation of calcification at day 21.

### Example 7: Rat Osteoarthritis Model

Lewis rats (n=40) underwent surgery to induce a medial meniscal tear and transect the collateral ligament in the right knee joint (27). Intra-articular injection of 50 μl vehicle (0.9% NaCl) (n=20) or peptide according to SEQ ID NO: 1 (20 μM; n=20) was performed on days 7, 10, 14, 17, 21 and 24 post-surgery. Gait analysis was performed at day 20 and necropsy was done at day 28 post-surgery. Histological sections of decalcified and paraffin embedded knee joints were obtained and stained with toluidine blue (28) for histological scoring (24). The experiment was performed and histological sections were stained at Bolder Biopath Inc., Boulder, USA.

### Example 8: Statistical Analyses

A two-tailed student t-test was used for comparisons of two groups. Data from precipitation assays was assumed to be normally distributed. For multiple group comparisons a one-way ANOVA with Dunnet's or Bonferroni post-test was used. For the rat OA model, groups were compared using a two-tailed student t-test for normal distributed data (D'Agostino-Pearson normality test) or a Mann-Whitney U test for data that was not normally distributed. Statistical analysis was done in Graphpad Prism 8.

### REFERENCES

1. Jaminon A, Reesink K, Kroon A, Schurgers L. The Role of Vascular Smooth Muscle Cells in Arterial Remodeling: Focus on Calcification-Related Processes. Int J Mol Sci. 2019; 20(22):5694.
2. Mulay S R, Anders H-J. Crystal nephropathies: mechanisms of crystal-induced kidney injury. Nature Reviews Nephrology. 2017; 13(4):226-40.
3. McCarthy G M, Dunne A. Calcium crystal deposition diseases—beyond gout. Nature Reviews Rheumatology. 2018; 14(10):592-602.
4. Parhami F, Bostr, ouml, m K, Watson K, Demer L L. Role of Molecular Regulation in Vascular Calcification. Journal of Atherosclerosis and Thrombosis. 1996; 3(2):90-4.
5. McCarty D. Crystals, joints, and consternation. Ann Rheum Dis. 1983; 42(3):243-53.
6. Mortensen J D, Baggenstoss A H. Nephrocalcinosis: A Review. American Journal of Clinical Pathology. 1954; 24(1):45-63.
7. Conway R, McCarthy G M. Calcium-Containing Crystals and Osteoarthritis: an Unhealthy Alliance. Current Rheumatology Reports. 2018; 20(3):13.
8. Molloy E S, McCarthy G M. How crystals damage tissue. Current Rheumatology Reports. 2004; 6(3):228-34.
9. Bäck M, Aranyi T, Cancela M L, Carracedo M, Conceição N, Leftheriotis G, et al. Endogenous Calcification Inhibitors in the Prevention of Vascular Calcification: A Consensus Statement From the COST Action EuroSoftCalcNet. Frontiers in Cardiovascular Medicine. 2019; 5(196).
10. Millán J L. The role of phosphatases in the initiation of skeletal mineralization. Calcif Tissue Int. 2013; 93(4):299-306.
11. Jafri L, Khan A H, Azeem S. Ionized calcium measurement in serum and plasma by ion selective electrodes: comparison of measured and calculated parameters. Indian J Clin Biochem. 2014; 29(3):327-32.
12. Goldstein D A. Serum Calcium. In: rd, Walker H K, Hall W D, Hurst J W, editors. Clinical Methods: The History, Physical, and Laboratory Examinations. Boston 1990.
13. Bansal V K. Serum Inorganic Phosphorus. In: rd, Walker H K, Hall W D, Hurst J W, editors. Clinical Methods: The History, Physical, and Laboratory Examinations. Boston 1990.
14. Okawa A, Nakamura I, Goto S, Moriya H, Nakamura Y, Ikegawa S. Mutation in Npps in a mouse model of ossification of the posterior longitudinal ligament of the spine. Nature Genetics. 1998; 19(3):271-3.
15. Harmey D, Hessle L, Narisawa S, Johnson K A, Terkeltaub R, Millán J L. Concerted regulation of inorganic pyrophosphate and osteopontin by akp2, enpp1, and ank: an integrated model of the pathogenesis of mineralization disorders. Am J Pathol. 2004; 164(4):1199-209.
16. Jansen R S, Küçükosmanoglu A, de Haas M, Sapthu S, Otero J A, Hegman I E M, et al. ABCC6 prevents ectopic mineralization seen in pseudoxanthoma elasticum by inducing cellular nucleotide release. Proc Natl Acad Sci USA. 2013; 110(50):20206-11.
17. Heiss A, DuChesne A, Denecke B, Grötzinger J, Yamamoto K, Renné T, et al. Structural Basis of Calcification Inhibition by α2-HS Glycoprotein/Fetuin-A: FORMATION OF COLLOIDAL CALCIPROTEIN PARTICLES. Journal of Biological Chemistry. 2003; 278(15):13333-41.
18. Schinke T, Amendt C, Trindl A, Pöschke O, Müller-Esterl W, Jahnen-Dechent W. The Serum Protein α2-HS Glycoprotein/Fetuin Inhibits Apatite Formation in Vitro and in Mineralizing Calvaria Cells: A POSSIBLE ROLE IN MINERALIZATION AND CALCIUM HOMEOSTASIS. Journal of Biological Chemistry. 1996; 271(34):20789-96.
19. Cai M M X, Smith E R, Holt S G. The role of fetuin-A in mineral trafficking and deposition. Bonekey Rep. 2015; 4:672-.
20. Ng S, Guo J, Ma J, Loo S C. Synthesis of high surface area mesostructured calcium phosphate particles. Acta biomaterialia. 2010; 6(9):3772-81.
21. Kapustin A N, Chatrou M L L, Drozdov I, Zheng Y, Davidson S M, Soong D, et al. Vascular Smooth Muscle Cell Calcification Is Mediated by Regulated Exosome Secretion. Circulation Research. 2015; 116(8):1312-23.
22. Jubeck B, Muth E, Gohr C M, Rosenthal A K. Type II collagen levels correlate with mineralization by articular cartilage vesicles. Arthritis Rheum. 2009; 60(9):2741-6.
23. Maniatopoulos C, Sodek J, Melcher A H. Bone formation in vitro by stromal cells obtained from bone marrow of young adult rats. Cell and Tissue Research. 1988; 254(2):317-30.
24. Pritzker K P H, Gay S, Jimenez S A, Ostergaard K, Pelletier J P, Revell P A, et al. Osteoarthritis cartilage histopathology: grading and staging. Osteoarthritis and Cartilage. 2006; 14(1):13-29.
25. Wisse E, Braet F, Duimel H, Vreuls C, Koek G, Olde Damink S W M, et al. Fixation methods for electron microscopy of human and other liver. World J Gastroenterol. 2010; 16(23):2851-66.
26. Reynolds J L, Joannides A J, Skepper J N, McNair R, Schurgers L J, Proudfoot D, et al. Human vascular smooth muscle cells undergo vesicle-mediated calcification in response to changes in extracellular calcium and phosphate concentrations: a potential mechanism for accelerated vascular calcification in ESRD. J Am Soc Nephrol. 2004; 15(11):2857-67.
27. Janusz M J, Bendele A M, Brown K K, Taiwo Y O, Hsieh L, Heitmeyer S A. Induction of osteoarthritis in the rat by surgical tear of the meniscus: Inhibition of joint damage by a matrix metalloproteinase inhibitor. Osteoarthritis Cartilage. 2002; 10(10):785-91.
28. Schmitz N, Laverty S, Kraus V B, Aigner T. Basic methods in histopathology of joint tissues. Osteoarthritis Cartilage. 2010; 18 Suppl 3:S113-6.

1. Circular polypeptide consisting of an amino acid sequence according to SEQ ID NO: 1, wherein the amino acids are D-amino acids.

2. Pharmaceutical composition comprising a polypeptide according to claim 1 and a pharmaceutically acceptable carrier.

3. Polypeptide according to claim 1 for use in the treatment of a disease selected from the group consisting of osteoarthritis, frozen shoulder syndrome, heterotopic ossification, vascular calcification, kidney stones and calcinosis.


SEQUENCE LISTING (1 sequence)

SEQ ID NO: 1 - protein, 30 aa, Homo Sapiens
LIYRQPNCDDPETEEAALVAIDYIAPHGPG